(19) United States
(12) Patent Application Publication
(10) Pub. No.: US 2015/0038945 A1
(43) Pub. Date: Feb. 5, 2015

(54) MODULAR SYSTEM FOR COLLECTING, MEASURING, STORING AND FEEDING BREAST EXPRESSED MILK

(71) Applicant: Jill C. McCabe, Apex, NC (US)

(72) Inventor: Jill C. McCabe, Apex, NC (US)

(21) Appl. No.: 14/340,751

(22) Filed: Jul. 25, 2014

Related U.S. Application Data
(60) Provisional application No. 61/860,922, filed on Aug. 1, 2013.

(51) Int. Cl. A61M 1/06

(52) U.S. Cl. CPC A61M 1/06; A61M 1/062
USPC 604/514; 604/74; 206/223

(57) ABSTRACT

A modular system operative to work in conjunction with a breast pump for collecting and managing the storage or dispensing of milk extracted from a woman's breast by the breast pump. The modular system includes a series of interchangeable components, including a series of sleeves and a variety of lids that can be secured to the sleeves to form containers for receiving and holding milk extracted by the breast pump.

### CROSS-REFERENCE TO RELATED APPLICATION

This application claims priority under 35 U.S.C. §119(e) from the following U.S. provisional application: Application Ser. No. 61/860,922 filed on Aug. 1, 2013. That application is incorporated in its entirety by reference herein.

### FIELD OF THE INVENTION

The present invention relates to a system for managing the collection and feeding of milk expressed from a woman's breast.

### BACKGROUND OF THE INVENTION

The advantages of breast-feeding babies is undeniable. Many mothers, because of work, travel or otherwise being separated from their baby, employ breast pumps to extract milk from their breasts. However, managing the collection, storage and feeding of the milk is challenging. There is a need for a system that is simple, easy to use and which will collect, meter, store and feed milk expressed via a breast pump in as few as one step. Moreover, some babies are not able to latch onto breasts and in these cases using a breast pump to provide milk for the baby is a viable option. Here again, there is a need for a system that is simple and easy to use for collecting and managing milk produced via a breast pump.

### BRIEF DESCRIPTION OF THE DRAWINGS

FIG. 1 is a view that illustrates the kit of the present invention which includes a series of components for managing the collection and dispensing of milk extracted from a woman's breast by a breast pump.

FIG. 2 is a schematic illustration of the modular system of the present invention being utilized with a breast pump to measure or meter milk extracted by the breast pump.

FIG. 2A is an exploded view of a portion of FIG. 2 showing two sleeves of the kit of the present invention and a valve assembly.

FIG. 2B is an exploded view of the valve assembly.

FIG. 3 is a view illustrating components of the kit being utilized for direct feeding.

FIG. 4 is a view that illustrates how components of the kit are used to contain and store milk extracted by a breast pump.

### DESCRIPTION OF EXEMPLARY EMBODIMENTS

With further references to the drawings, there is shown therein a modular system for managing the collection and feeding of milk extracted by a breast pump. The modular system is designed to operate in conjunction with a breast pump. As will be appreciated from subsequent portions of this disclosure, the modular system described herein is operative to collect, meter, feed and store milk extracted by a breast pump.

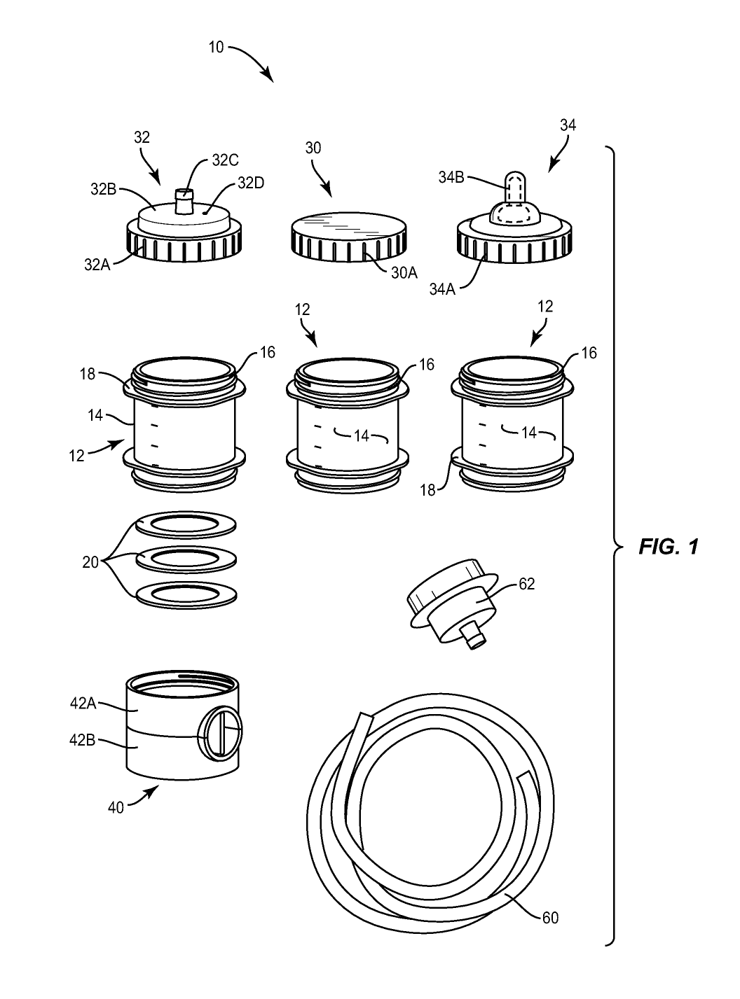

As shown in FIG. 1, the modular system for managing the collection of expressed milk can be provided in a kit form. In one example, the kit includes a series of sleeves, various lids for connecting to the sleeves to form containers, and a valve assembly. As will be appreciated from subsequent portions of the disclosure, the valve assembly is utilized to interconnect two sleeves together and to isolate one sleeve from the other in order that one sleeve can be employed to measure or meter milk extracted by the breast pump. This allows exact measured feeding and reduces the risk of contaminating the milk transferred from one sleeve to another sleeve via the valve assembly.

With particular reference to the kit shown in FIG. 1, the modular milk collection system is shown therein and indicated generally by the numeral 10. The kit includes two or more sleeves with each sleeve being referred to generally by the numeral 12. Sleeve 12 can be constructed of various suitable materials such as plastic or glass and can be of various sizes. Each sleeve includes a cylindrical wall 14. In one embodiment, the cylindrical wall 14 is transparent. Cylindrical wall 14 includes opposed open ends. Formed on each end of the cylindrical wall 14 is a threaded end 16. Disposed inwardly of each threaded end 16 is an annular flange 18. In some embodiments, a seal ring 20 is secured to or integral with the outer ends of the sleeve 12. In one embodiment, at least one of the sleeves 12 is adapted to measure a volume of milk contained therein. In order to facilitate this feature of the modular milk collection system, the cylindrical wall 14 is provided with indicia for indicating the volume of milk contained within a container formed in part by the sleeve 12.

Continuing to refer to FIG. 1, the kit includes a series of threaded lids. Each of the lids is designed to connect to an end portion of one of the sleeves 12. Each of the lids includes internal threads for securing to the threaded ends 16 of the sleeves 12. In the exemplary kit shown in FIG. 1, there are three types of lids provided. In other embodiments, the kit may be provided with one or more different lids. Kit 10 includes one or more closed lids with each closed lid being indicated generally by the numeral 30. In addition, the kit includes one or more lids having an inlet opening. This lid is indicated generally by the numeral 32. Finally, the kit includes one or more lids that include a nipple, this lid being indicated generally by the numeral 34.

As seen in FIG. 1, the closed lid 30 includes a twist rim 30A. Internally within the twist rim 30A there is provided threads that are configured to threadedly engage the threaded end 16 of the sleeve 12. In some cases, the closed lid 30 may form the bottom of a container that is formed by connecting two lids to a sleeve 12. See FIG. 4. Thus, in the embodiment illustrated herein, the closed lid 30 includes a generally flat surface that enables the formed container to be situated in a stable upright position when the closed lid forms the bottom of the container. Lid 32 also includes a twist rim 32A and a raised section 32B. Formed in the raised section 32B is an inlet (stub tube) 32C. There is also provided an air outlet valve 32D. This may include a one-way check valve that allows air to escape from a sleeve through the lid when milk is being directed into the sleeve. The third lid is referred to as a nipple lid 34. It also includes a twist section 34A that, like the other lids, includes an internal thread for threading onto the threaded end 16 of the sleeve 12. In addition, the nipple lid 34 includes a nipple 34B.

The kit shown in FIG. 1 also includes a valve assembly indicated generally by the numeral 40. Various different valve designs can be provided to accomplish the function intended for the valve. As will be discussed hereafter, valve assembly 40 is utilized between two sleeves 12 and enables one sleeve to be isolated from the other for the purpose of measuring a volume of milk inside the sleeve. Once the volume of milk has been measured, the valve can be opened such that the measured volume of milk is allowed to flow from one sleeve to the other sleeve.

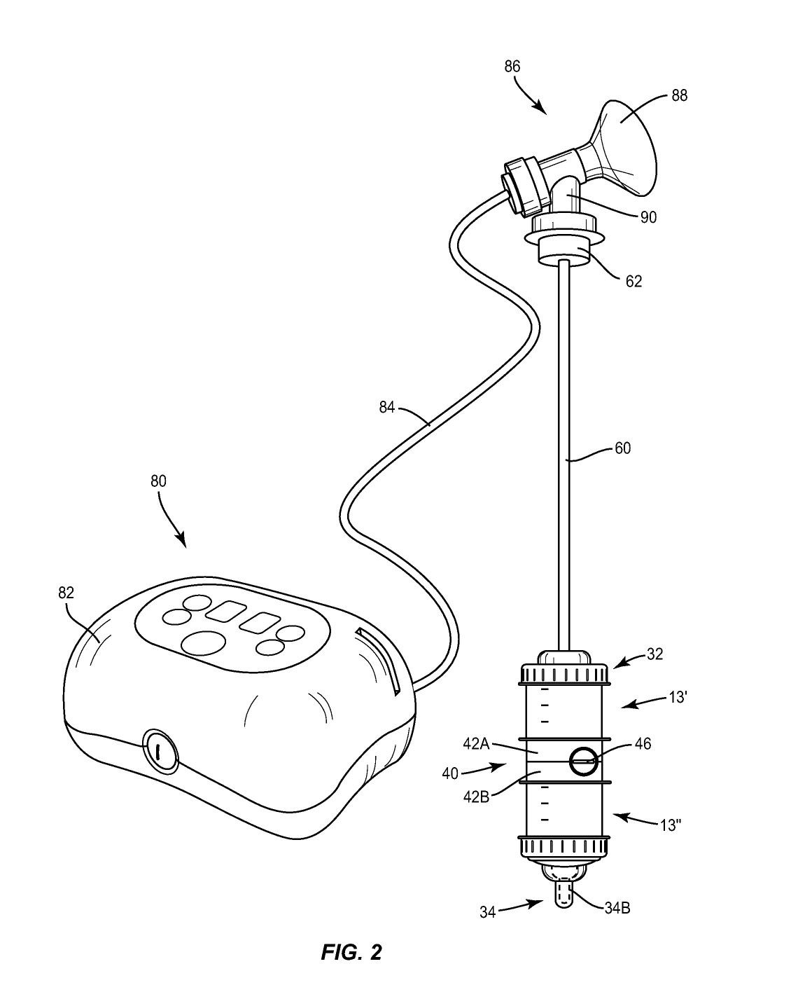

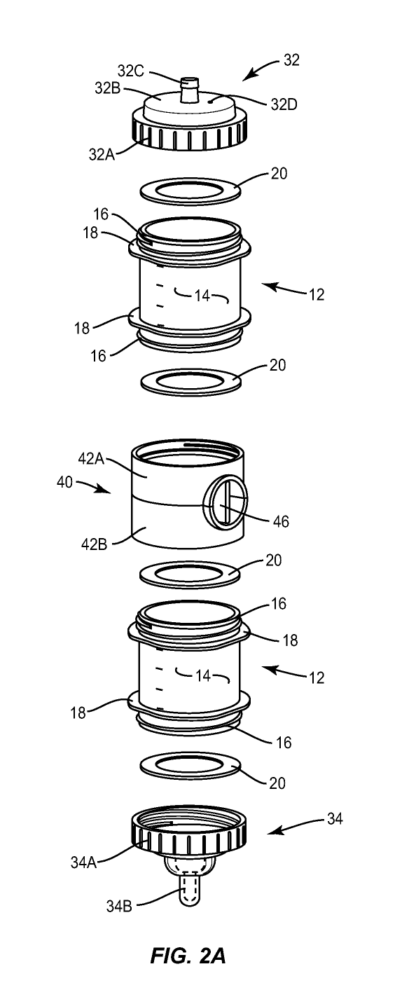

Viewing valve assembly 40 in more detail, it is seen that the valve assembly includes a housing structure that is formed by two sections, section 42A and section 42B. Both of these sections are threaded such that the housing of the valve assembly 40 can be interconnected between two sleeves 12. That is, the internal threads formed in one section of the housing can be screwed onto the external threads 16 formed on an end portion of one sleeve, and internal threads provided in the other section of the housing can be screwed onto external threads formed on an end portion of another sleeve 12. Consequently, the valve assembly 40 can be easily connected between two sleeves 12. See FIG. 2.

The housing (42A and 42B) includes a through opening which enables milk to pass through the through opening. A ball-type valve is positioned in the through opening so as to open and close the through opening. In particular, as viewed in FIG. 2B, a ball 44 is seated in the through opening. Ball 44 includes an opening 44A that extends completely through the ball. A manual actuator 46 is secured to the ball 44 and projects therefrom. As seen in FIGS. 2 and 2A, the manual actuator 46 projects from the side of the valve assembly 40 such that it can be easily accessed. As noted above, ball 44 is seated in a structure in the through opening of the valve assembly 40. The structure for seating the valve includes a seat 48. There is a valve body 50 that is disposed over the ball 44 and contained within the housing. Underlying the ball 44 is a valve cap 52. The valve cap 52 is secured to the valve seat 48 through a treaded connection. Both the valve seat 48 and valve cap 52 are secured within the housing of the valve assembly. Both the valve body 50 and valve cap 52 include an opening for permitting milk to flow through these structures.

In an open position, ball 44 is oriented such that its opening 44A is aligned with the openings in the valve body 50 and valve cap 52, as well as the through opening in the valve assembly 40. This permits milk to flow through the valve assembly 40. By rotating the manual actuator 46, the opening 44A of the ball 44 is turned such that it does not align with the through opening in the valve assembly or with the openings in the valve body 50 or valve cap 52. Effectively the ball 44 creates a sealed relationship in the valve assembly 40 and effectively closes such that milk cannot pass through the valve assembly.

Continuing to refer to the kit shown in FIG. 1, the modular system 10 is provided with means for channeling milk from a breast pump into a container. A container is formed by a sleeve 12 and two of the lids, or in one case a container is formed by a sleeve and one of the lids and the valve assembly 40. In the embodiment illustrated herein, there is provided a flexible tube 60 that is designed to connect to the stub tube 32C that extends from the lid 32. As will be discussed subsequently herein, the flexible tube 60 is designed to channel milk from the breast pump into one container.

Finally, the kit shown in FIG. 1 is provided with an adapter 62 that is designed to attach between an outlet of a breast pump and the flexible tube 60. The design of the adapter 62 may vary. This is because different breast pumps will require different adapters. In any event, the function of the adapter 62 is to provide an interface or an interconnection between an outlet of a breast pump and the means for directing milk into one of the containers which, in this case, is the flexible tube 60.

The components shown in FIG. 1 are exemplary components for one embodiment of the kit. It is appreciated by those skilled in the art that the number of sleeves, lids, seals, etc. can vary. For example, the kit may include many closed lids 30 and numerous lids 32 and 34. In addition, the kit may include more than three sleeves.

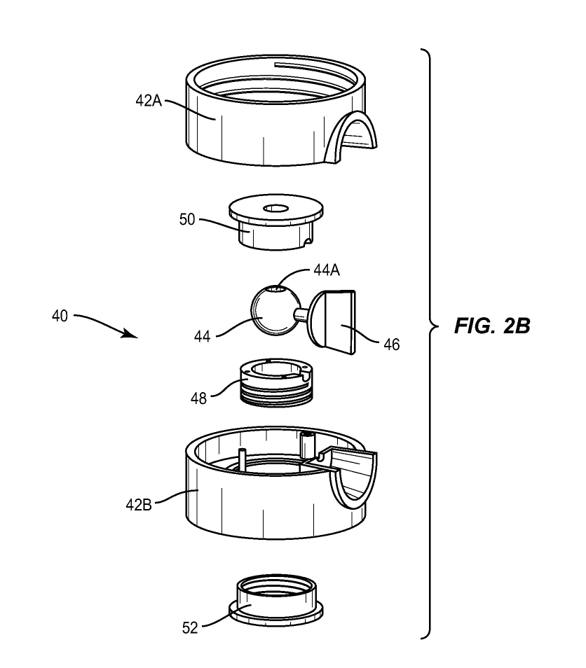

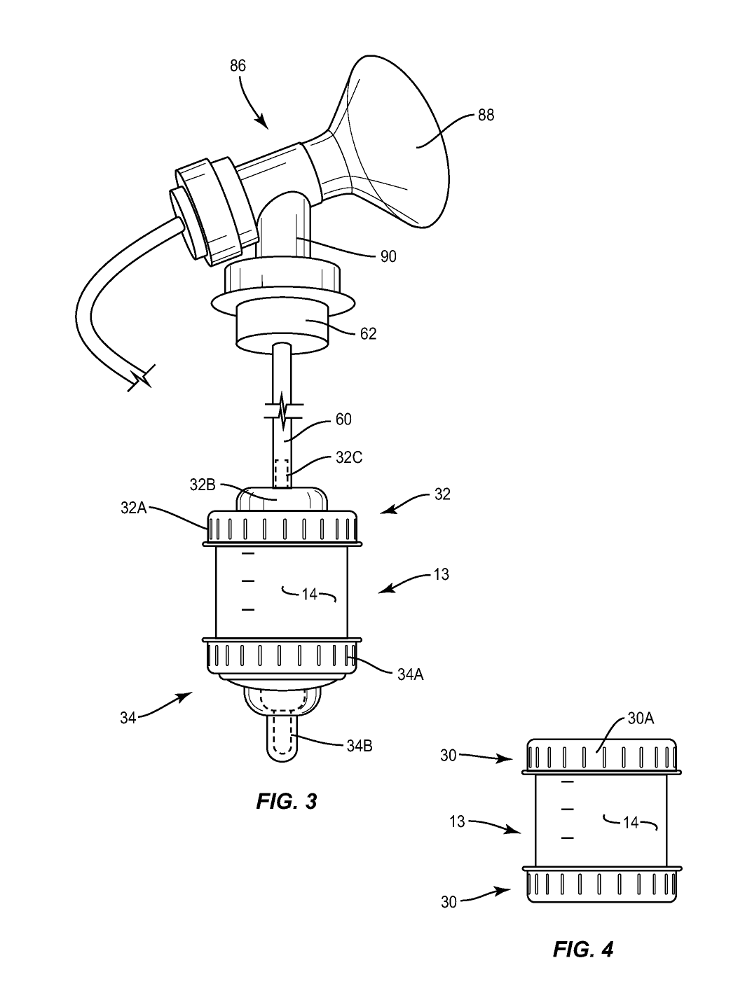

The modular system 10 for managing the collection of expressed milk is designed to work with a breast pump. The breast pump can be any make or model of commercially available breast pumps. For example, the breast pump may be a manual pump, a battery powered pump, or an electric powered pump. A representation of a breast pump is shown in FIG. 2. The breast pump shown there is indicated generally by the numeral 80. Breast pump 80 includes a control unit 82 including an internal pump for generating a vacuum. A vacuum line 84 extends from the control unit 82 to a flange assembly indicated generally by the numeral 86. Flange assembly 86 includes a breast flange 88 and an outlet 90 which may or may not be threaded. As discussed above, the sleeves 12 and the various lids 30, 32 and 34, can be used to form containers indicated generally by the numeral 13. For example, a container 13 can be formed by securing two closed lids 30 to opposite ends of a sleeve 12. See FIG. 4. In addition, a container can be formed by securing a closed lid 30 to one end of a sleeve and the lid 32 having the inlet opening to the other end of the sleeve. Also, a container or bottle having a nipple can be formed. This occurs by securing the nipple lid 34 to one end of a sleeve 12 and the closed lid 30 or the lid 32 to the other end of the sleeve. See FIG. 3. Thus, it is appreciated that various combination of lids can be secured to the sleeves 12 to form various configured containers or bottles 13.

The modular system shown in FIG. 1 can be used in various ways to manage milk produced through breast feeding. As will be described hereafter, the system can be utilized to measure volumes of milk emitted by the breast pump 80. Once a measured amount of milk is determined, the measured volume can be transferred to a container 13 via the valve assembly 40. In another mode of operation, the modular system 10 can be used to directly feed a baby. That is, the system can be employed to feed milk to a baby through a container 13 that is being supplied milk by the breast pump 80. A third way of utilizing the modular system 10 of the present invention is that a container 13 can be communicatively connected to the outlet of the breast pump 80 to receive milk for storage.

Turning to FIG. 2, it is seen that two containers, 13′ and 13″, are connected by the valve assembly 40. The upper container 13′, shown in FIG. 2, may be termed a measuring container or a metering container. With the valve assembly 40 closed, the breast pump 80 is operative to pump milk from the breast of a woman through the outlet 90 of the flange assembly 86 and through the adapter 62 into the flexible line 60. Milk in the flexible line 60 is directed through the lid 32 into the upper (as viewed in FIG. 2) container 13′. As the breast pump 80 operates, milk accumulates in the upper container 13′. Once a selected volume of milk has accumulated in the upper container 13′, that volume of milk can be transferred to the lower container 13″ by opening the valve assembly 40. In some instances, the lower container 13″ may be referred to as a collection container. Once transferred to the lower container 13″, the valve assembly 40 can be closed and the milk in the lower container 13″ can be fed to the baby. If desired, the breast pump 80 can be continued to be operated to pump milk into the upper container 13′ while the baby is consuming milk from the lower container 13″. There is also another option. For example, at any time once a selected volume of milk has been accumulated in the upper container 13′, the breast pump 80 can be deactivated and the upper container 13″ can be converted to a storage container where the closed lid 30 is placed on both ends of the container.

In another mode of use, one container 13 can be utilized to directly feed a baby while milk is being pumped into the container. This is illustrated in FIG. 3. Note that one end of the container 13 includes a nipple lid 34 that includes a nipple 34B. The other end of the container includes a lid 32 that includes an opening for permitting milk to flow through the lid. In this case, the flexible tube 60 is operatively interconnected between the adapter 62 and the stub tube 32C of the lid 32. Breast pump 80 is activated, causing a vacuum to be created about the breast flange 88 which results in milk being drawn from the breast of a woman through the outlet 90, through the adapter 62 and on through the flexible tube 60 into the container 13. During this time while the breast pump 80 is pumping milk into the container 13, the baby is consuming milk via the nipple 32B at the same time.

Another mode of operation is where the modular milk collection system 10 is utilized to collect milk for storage. This is illustrated in FIG. 4. In this case, the container 13 has a closed lid 30 secured to one end thereof, the bottom end. Secured to the other end is lid 32 having the inlet opening for permitting milk to pass through the lid. In this case, the breast pump 80 is actuated and causes milk to flow through the flexible tube 60, through the lid 32 into the container 13 where milk accumulates. Once a selected volume of milk has accumulated in the container 13, the breast pump 80 can be deactivated and the lid 32 can be replaced with a closed lid 30. See FIG. 4. Now the container 13 is suitable for storage and can be placed in a refrigerator or other controlled environment for future use.

From the foregoing discussion, it is appreciated that the modular milk collection system 10 is easy to use and has many advantages. The system and uses described above make breast feeding a practical and reasonable option for many women that would not otherwise have that option. The modular system for managing the collection, storage and dispensing of expressed milk enables a baby to be fed directly from the breast pump 80 or enables breast milk to be collected from the breast pump and thereafter stored for future use. In another embodiment, the modular expressed milk management system can be utilized to measure or meter milk supplied by the breast pump 80 and thereafter be directly transferred to a container which may be used to directly feed the baby. The parts and components of the modular system can be easily interchanged and rearranged to carry out various functions and to provide numerous breast feeding options.

The present invention may, of course, be carried out in other ways than those specifically set forth herein without departing from essential characteristics of the invention. The present embodiments are to be considered in all respects as illustrative and not restrictive, and all changes coming within the meaning and equivalency range of the appended claims are intended to be embraced therein.

What is claimed is:

1. A kit for managing milk extracted from a woman's breast by a breast pump, the kit comprising:
a series of sleeves having open ends;
a series of differently configured lids for connecting to opposite ends of the sleeves;
a valve assembly configured to interconnect two sleeves, the valve assembly being moveable between open and closed positions wherein in the open position the valve is operative to permit milk to flow from one sleeve through the valve assembly into the other sleeve; and
a milk conveying line for connecting to one lid and directing milk from the breast pump through the lid and into one of the sleeves of the kit.

2. The kit of claim 1 wherein each sleeve includes a surrounding wall; threads formed around opposite ends of the surrounding wall; wherein each lid includes threads configured to screw on and screw off the threads formed on opposite ends of the surrounding wall of each sleeve; and at least one of the lids of the kit including an opening formed therein for permitting milk to pass through the opening.

3. The kit of claim 1 wherein each sleeve includes a surrounding wall; and a pair of spaced apart annular flanges that extend around the surrounding wall and project outwardly therefrom.

4. The kit of claim 3 including an exterior thread extending around the surrounding wall outwardly of each annular flange; and wherein each lid includes an internal thread that is configured to engage the exterior threads on the surrounding wall such that the lid can be secured to the surrounding wall.

5. The kit of claim 1 further including at least one adapter configured to interconnect the breast pump with the milk conveying line such that milk flowing from the breast pump flows through the adapter and into the milk conveying line.

6. The kit of claim 1 wherein the kit includes a breast pump.

7. The kit of claim 1 wherein the lids of the kit include a nipple lid having a nipple, a closed lid, and a lid having an opening that permits milk to flow through the lid.

8. The kit of claim 1 wherein the valve assembly includes a housing that includes two sets of threads wherein each set of threads is configured to threadedly engage threads formed on opposed end portions of each sleeve, thereby permitting the valve assembly to be interconnected between two sleeves.

9. The kit of claim 1 including a breast pump; at least one adapter configured to interconnect the breast pump with the milk conveying line such that milk flowing from the breast pump flows through the adapter and into the milk conveying line; and wherein the lids of the kit include a nipple lid having a nipple, a closed lid, and a lid having an opening for permitting milk to flow through the opening in the lid.

10. The kit of claim 9 wherein at least one sleeve includes two sets of external threads formed on opposite end portions of the sleeve; and wherein at least one of the lids of the kit includes internal threads that are configured to threadedly engage the exterior threads on opposed end portions of the sleeve.

11. A device for collecting milk from a breast pump comprising:
a measuring container having indicia for indicating the volume of milk contained in the measuring container;
an inlet associated with a measuring container and operative to direct milk from the breast pump into the measuring container;
a collection container for collecting milk from the measuring container;
a valve assembly interposed between the measuring container and the collection container and wherein both the measuring container and collection container are connected to the valve assembly through liquid-tight connections; and
the valve assembly movable between open and closed positions wherein, in the open position, milk in the measuring container flows from the measuring container through the valve assembly into the collection container and, in the closed position, milk in the measuring container is precluded from flowing through the valve assembly into the collection container.

12. The device of claim 11 wherein the measuring container and the collection container are aligned.

13. The device of claim 11 wherein the measuring container, collection container and valve assembly are all axially aligned.

14. The device of claim 11 wherein the measuring container includes an inner end portion and the collection container includes an inner end portion, and wherein the valve assembly includes opposed outer end portions and wherein the outer end portions of the valve assembly are threadedly connected to the inner end portions of the measuring container and collection container.

15. The device of claim 11 wherein the collection container includes an outer end portion that is configured to receive a nipple.

16. The device of claim 11 wherein the measuring container or collection container is convertible to a direct feed container having a nipple on a first end and a feed tube extending from a second end and wherein the direct feed container is configured to be operatively connected to a breast pump such that milk is delivered from the breast pump to the direct feed container while a baby consumes milk from the direct feed container.

17. The device of claim 11 where the valve assembly includes a housing having a through opening; and a valve seated in the housing for opening and closing the through opening.

18. The device of claim 17 wherein the valve assembly includes an manual actuator operative to move the valve between open and closed positions and wherein the manual actuator projects outwardly from the valve where it can be manipulated by hand.

19. The device of claim 11 wherein the valve assembly includes a housing, a valve seated in a valve body and moveable between open and closed positions; and a manual actuator operatively connected to the valve for moving the valve between the open and closed positions.

20. The device of claim 11 including a breast pump for directing milk into the inlet associated with the measuring container.

21. A method of directly feeding a baby with a breast pump and a milk collection container comprising: pumping milk from a woman's breast with a breast pump; directing the milk from the breast pump through an elongated flexible tube to a milk holding container; and feeding milk from the milk holding container through a nipple to a baby while pumping milk from the woman's breast with the breast pump and directing the pumped milk through the elongated flexible tube into the milk holding container.

22. The method of claim 21 including pumping the milk from the breast pump to a measuring container and thereafter transferring the milk to the milk holding container.

23. The method of claim 21 wherein prior to reaching the milk holding container, the milk is pumped into and through a measuring container and thereafter passes through a valve assembly interconnected between the measuring container and the milk holding container.

24. The method of claim 21 including connecting one end of the flexible tube to an adapter and connecting the adapter to the breast pump and pumping milk from the woman's breast through the adapter and into the elongated flexible tube.
